(19) United States
(12) Patent Application Publication
(10) Pub. No.: US 2008/0031869 A1
(43) Pub. Date: Feb. 7, 2008

(54) Pain relief composition

(76) Inventor: Juliette S. Fontaine, Clinton, MD (US)

Correspondence Address: LITMAN LAW OFFICES, LTD., P.O. BOX 15035, CRYSTAL CITY STATION, ARLINGTON, VA, 22215, US

(21) Appl. No.: 11/882,572

(22) Filed: Aug. 2, 2007

Related U.S. Application Data
(60) Provisional application No. 60/834,741, filed on Aug. 2, 2006.

(51) Int. Cl. A61K 38/48; A61K 31/201; A61K 31/34; A61K 31/352; A61P 25/00; A61K 31/415; A61K 31/7028; A61K 33/30

(52) U.S. Cl. 424/94.65; 424/641; 514/27; 514/400; 514/456; 514/474; 514/560

(57) ABSTRACT

The pain relief composition is a combination of dietary and nutritional supplements that has been found effective for the relief of pain arising from a wide variety of diseases and injuries, regardless of severity or chronicity. The composition is formulated for oral administration, such as a tablet, soft gel capsule, liquid, or the like. The pain relief composition includes: at least one omega fatty acid; at least one antioxidant; and at least one amino acid.

### CROSS-REFERENCE TO RELATED APPLICATION

This application claims the benefit of U.S. Provisional Patent Application Ser. No. 60/834,741, filed Aug. 2, 2006.

### BACKGROUND OF THE INVENTION

1. Field of the Invention

The present invention relates to nutritional and dietary supplements, and particularly to a pain relief composition formed from a combination of nutritional and dietary supplements.

2. Description of the Related Art

Pain is a symptom that may accompany a wide variety of diseases and traumatic injuries. Sometimes the degree of pain may be correlated with physical signs that are correlated with the extent of the disease or the severity of the injury, but quite often there is no such observable correlation. This phenomenon reflects that the pathology and mechanism by which the body senses pain is still imperfectly understood.

The traditional approach by the medical community has been to repair or excise the physical damage, immobilize painful joints, and prescribe drugs, such as analgesics, narcotics and anti-inflammatory medications for the relief of pain, often accompanied by antibiotics. Prescription medications are often accompanied by massages, physical therapy, and either the application of heat for muscle strain or ice for the reduction of edema or swelling. In recalcitrant cases, pain is sometimes treated by electrical stimulation, such as a TENS stimulator.

While such forms of treatment are often effective, frequently pain will prove to be intractable or unresponsive to conventional forms of treatment. Even when effective, such treatment modalities are often accompanied by undesirable side effects, including: narcotic addiction; decreased ability to perform routine daily activities, such as work and driving; interference with sleep patterns; and physical side effects, such as increased heart rate or blood pressure, fatigue, polyuria or constipation, decreased concentration, or dexterity, imbalance, and other cardiovascular, pulmonary, gastrointestinal, and central nervous system symptoms.

Consequently, there has been some research directed towards alternative approaches to the relief of pain. Statistical studies have shown that individuals having similar impairments and comparable levels of pain often respond differently to the same conventional course of treatment and have shorter or longer course of treatment depending upon dietary considerations and shortages or imbalances in the levels of certain nutrients, minerals, enzymes and amino acids. In addition, certain traditional remedies derived from herbs, plants, animals, and other natural sources have reputedly been used with success by Native American, Chinese, Indian, and other civilizations. It has sometimes been possible to identify an active ingredient in such natural sources for use with particular impairments; however, more often researchers have deduced that the benefit derived from dietary and nutritional sources results from combinations of ingredients, often found on an empirical basis and whose mechanism is only imperfectly understood.

However, there remains a need for a pain relief composition derived from dietary and nutritional supplements that is effective in alleviating or promoting the relief of pain, whether mild, moderate, severe, acute or chronic, and that is also free from the undesirable side effects of traditional pharmaceuticals and medications. Thus, a pain relief composition solving the aforementioned problems is desired.

### SUMMARY OF THE INVENTION

The pain relief composition is a combination of dietary and nutritional supplements that has been found effective for the relief of pain arising from a wide variety of diseases and injuries, regardless of severity or chronicity. The composition is formulated for oral administration, such as a tablet, soft gel capsule, or the like, or may be given in liquid form. The pain relief composition comprises: at least one omega fatty acid; at least one antioxidant; and at least one of Vitamin C, bioflavonoid, calcium, magnesium and vitamins.

In a first embodiment, the composition comprises: about 100 mg to 2,000 mg of omega fatty acids, such as flaxseed oil or a combination of eicosapentaenoic acid (EPA) and gamma-linolenic acid (GLA); about 500 mg quercetin; about 250-500 mg rutin; about 1,000 mg of other mixed bioflavonoids; about 1,000 mg Vitamin C; about 500 mg calcium; about 250 mg magnesium; about 15 mg zinc; an effective amount of Vitamins B₁, B₂, B₆ and B₁₂; an effective amount of DL-phenylalanine; an effective amount of (MSM); and an effective amount of malic acid.

In a second embodiment, the composition comprises: about 100 to 2,000 mg of omega fatty acids, such as flaxseed oil or a combination of eicosapentaenoic acid (EPA) and gamma-linolenic acid (GLA); about 500 mg quercetin; about 250 mg rutin; about 50-1,000 mg of other mixed bioflavonoids; an effective amount of bromelain; about 100-1,000 mg Vitamin C; about 500 mg calcium; about 250 mg magnesium; about 10-15 mg zinc; an effective amount of Vitamins B₁, B₂, B₆ and B₁₂; an effective amount of DL- or L-phenylalanine; an effective amount of (MSM); and an effective amount of malic acid.

In a third embodiment, the composition comprises: about 1,000-2,000 mg of either EPA/GLA or S-adenosylmethionine (SAM)/GLA; about 500 mg calcium; about 250 mg magnesium; about 10 mg zinc; about 1,000 mg Vitamin C; about 500 mg quercetin; about 250 mg rutin; about 1,000 mg of other mixed bioflavonoids; about 300 mg bromelain; about 500 mg DL-phenylalanine; about 500 mg arginine; about 250 mg glutamine; about 25-75 mg aloe vera gel; about 200-5,000 IU Vitamin A; about 50-500 IU Vitamin D; and about 100 mg taurine.

The above pain relief compositions have been found to be effective in alleviating pain for such conditions as a fractured vertebra, a ruptured disc, toothache, and arthritis, and is believed to be effective for the relief of pain resulting from virtually any disease or injury that produces pain.

These and other features of the present invention will become readily apparent upon further review of the following specification.

### DETAILED DESCRIPTION OF THE PREFERRED EMBODIMENTS

The present invention is a pain relief composition formed from dietary or nutritional supplements that helps to reduce or alleviate pain associated with acute and chronic diseases or injuries, and is effective for mild, moderate and severe pain levels. The pain relief composition comprises: at least one omega fatty acid; at least one antioxidant; and at least one amino acid. The composition may also include minerals, vitamins, and enzymes that coact with the omega fatty acid, antioxidant, and amino acid to provide for effective relief of pain.

In a first embodiment, the composition comprises: about 100 mg to 2,000 mg omega fatty acids, such as flaxseed oil or a combination of eicosapentaenoic acid (EPA) and gamma-linolenic acid (GLA); about 10 mg to 1,000 mg quercetin, preferably about 500 mg; about 250 mg to 500 mg rutin, preferably about 250 mg; about 10 mg to 4,000 mg of other mixed bioflavonoids, preferably about 1,000 mg; about 10 mg to 5,000 mg Vitamin C, preferably about 1,000 mg; about 10 mg to 1,500 mg calcium, preferably about 500 mg; about 10 mg to 1,000 mg magnesium, preferably about 250 mg; about 5 mg to 50 mg zinc, preferably about 15 mg; an effective amount of Vitamins B₁, B₂, B₆ and B₁₂, preferably about 10 mg to 1,000 mg; about 10 mg to 2,000 mg of at least one essential amino acid selected from the group consisting of L-glutamine, DL-phenylalanine, L-phenylalanine, histidine, valine, taurine, lysine, glycine, arginine, leucine, and isoleucine, preferably an effective amount of DL-phenylalanine; an effective amount of (MSM), preferably about 50 to 3,000 mg; and an effective amount of malic acid, preferably about 10 mg to 1,000 mg.

Fats and fatty acids are necessary for good health. The body can make most fats and fatty acids, but cannot make two of the polyunsaturated fats, linoleic acid and alpha-linolenic acid, which are omega-6 and omega-3 fatty acids, respectively, because the body cannot add a carbon-carbon double bond more than nine carbons away from the carboxyl end of the molecule. For the same reason, the body cannot make longer chain derivatives of the essential fatty acids. Consequently, omega-6 and omega-3 fatty acids must be provided through the diet.

Flaxseed oil is a natural source of omega-6 and omega-3 fatty acids, in particular, linoleic and alpha-linolenic acid, which the body can subsequently convert to other fatty acids known to have anti-inflammatory effect, such as the omega-6 fatty acid gamma-linolenic acid (GLA) and the omega-3 fatty acid cis-5,8,11,14,17 eicosapentaenoic acid (EPA). EPA is also thought to be the precursor of a class of eicosanoids, including prostaglandins and leukotrienes, which have the effect of suppressing neutrophils and tumor necrosis factor, thereby reducing the immune system response including edema and tissue destruction, with consequent reduction in pain. Fish oil is a natural source of EPA. Commercial preparations of EPA/GLA are available in a ratio up to 10:1 that are suitable for use in the compositions of the present invention (the GLA may be derived from oil of evening primrose, which has a high GLA content as its primary active ingredient). Omega fatty acids have been found to be effective for the reduction of inflammation and pain in rheumatoid arthritis, heart impairments, etc.

Quercetin is a bioflavonoid responsible for the color of many fruits and vegetables. It has been found useful in reducing inflammation in arthritis, as well as the improvement of symptoms in fibromyalgia. Quercetin is also an effective antioxidant, reducing free radicals, such as O₂⁻, H₂O₂, and OH⁻. Free radicals may have a very damaging effect on polyunsaturated fatty acids, such as omega-6 and omega-3 fatty acids, by oxidizing the double bonds. Quercetin therefore has value in the present composition both for its primary anti-inflammatory effect, and also for preventing damage to the omega-3 fatty acids.

Rutin is a bioflavonoid, specifically a glycoside comprising quercetin and the disaccharide rutinose. Rutin is found in buckwheat and other plants, as well as in black tea ad apple peels. Rutin exhibits the same antioxidant properties as quercetin, and has also been found to exhibit anti-inflammatory activity.

Bioflavonoids is a large class of compounds, which, in addition to quercetin and rutin, includes catechins, kaempferol, hesperidin, anthrocyanidins, and other compounds found in various fruits, particularly citrus fruits, green tea, and rose hips. Bioflavonoids are known for their antioxidant properties, and therefore prevent degradation of the omega-3 fatty acids, as described above. Bioflavonoids also serve to protect Vitamin C.

Vitamin C (ascorbic acid) is known to strengthen capillaries and cell walls, and is crucial to the formation of collagen. Calcium, magnesium, zinc, and the B Vitamins, particularly Vitamin B₆, are required for metabolism of the omega-3 fatty acids, being used in conjunction with enzymes for the conversion of the fatty acids to useful derivatives.

L-phenylalanine is an essential amino acid found in most protein-rich food sources, such as dairy products, almonds, etc. L-phenylalanine is utilized by the body to synthesize tyrosine, which the body uses to make the neurotransmitters dopamine and norepinephrine, as well as epinephrine and serotonin. Tyrosine is also believed to be involved in the formation of certain enkephalins that have pain-relieving effects in the body. D-phenylalanine is an enantiomer of L-phenylalanine that is believed to have pain-killing properties. DL-phenylalanine is a 50-50 racemic mixture of the two enantiomers that has been shown to have pain-relieving effect in the treatment of osteoarthritis and rheumatoid arthritis.

Methylsulfonylmethane (MSM) is an organic compound that occurs in many fruits, vegetables, and grains. MSM is believed to result in pain relief, particularly in arthritis.

Malic acid is found in apples and other fruits. Malic acid, in combination with magnesium, is believed to provide relief for fibromyalgia and chronic fatigue syndrome.

In a second embodiment, the composition comprises: about 100 mg to 2,000 mg omega fatty acids, such as flaxseed oil or a combination of eicosapentaenoic acid (EPA) and gamma-linolenic acid (GLA), preferably about 1,000 to 2,000 mg; about 10 mg to 1,000 mg quercetin, preferably about 500 mg; about 10 mg, to 1,000 mg rutin, preferably about 250 mg; about 10 mg to 2,000 mg of other mixed bioflavonoids, preferably about 1,000 mg; an effective amount of bromelain, preferably about 10 mg to 1,000 mg; about 10 mg to 3,000 mg Vitamin C, preferably about 500-1,000 mg; about 10 mg to 1,500 mg calcium, preferably about 500 mg; about 10 mg to 1,000 mg magnesium, preferably about 250 mg; about 5 mg to 50 mg zinc, preferably about 15 mg; an effective amount of Vitamins B₁, B₂, B₆ and B₁₂, preferably about 10 mg to 1,000 mg; about 10 mg to 2,000 mg of at least one essential amino acid selected from the group consisting of L-glutamine, DL-phenylalanine, L-phenylalanine, histidine, valine, taurine, lysine, glycine, arginine, leucine, and isoleucine, preferably an effective amount of DL- or L-phenylalanine; an effective amount of (MSM), preferably about 50 mg to 3,000 mg; and an effective amount of malic acid, preferably about 10 mg to 1,000 mg.

The second embodiment of the composition is essentially identical to the first embodiment, with the exception that L-phenylalanine may optionally be used in place of DL-phenylalanine, and for the addition of bromelain. Bromelain is a group of enzymes containing sulfur that is derived from the pineapple plant, and is known to have anti-inflammatory effect. Bromelain is particularly known to exercise a synergistic effect with quercetin, enhancing the anti-inflammatory activity of both compounds.

In a third embodiment, the composition comprises: about 10 mg to 6,000 mg of either EPA/GLA or S-adenosylmethionine (SAM)/GLA, preferably about 1,000 mg; about 10 mg to 1,500 mg calcium, preferably about 500 mg; about 10 mg to 1,000 mg magnesium, preferably about 250 mg; about 5 mg to 50 mg zinc, preferably about 10 mg; about 10 mg to 6,000 mg Vitamin C, preferably about 1,000 mg; about 10 mg to 1,000 mg quercetin, preferably about 500 mg; about 10 mg to 1,000 mg rutin, preferably about 100 mg; about 10 mg to 4,000 mg of other mixed bioflavonoids, preferably about 1,000 mg; about 10 mg to 1,000 mg bromelain, preferably about 300 mg; about 10 mg to 2,000 mg of at least one essential amino acid selected from the group consisting of L-glutamine, DL-phenylalanine, L-phenylalanine, histidine, valine, taurine, lysine, glycine, arginine, leucine and isoleucine, preferably about 500 mg DL-phenylalanine, about 500 mg arginine, about 250 mg glutamine, and about 100 mg taurine; about 25 mg to 1,000 mg aloe vera gel, preferably about 75 mg; about 5 IU to 5,000 IU Vitamin A, preferably about 200 IU; about 5 IU to 500 IU Vitamin D, preferably about 50 IU; and about 25 mg to 1,000 mg flax seed oil, preferably about 1,000 mg.

S-adenosylmethionine (SAM) is a naturally occurring compound that plays a role in the immune system, maintains cell membranes, and is involved in the production and catabolism of several neurotransmitters, including serotonin, melatonin and dopamine, as well as Vitamin B₁₂. Studies have shown that SAM provides pain relief in osteoarthritis, and alleviates the symptoms of fibromyalgia, although the mechanism is not clear.

Arginine is an alpha-amino acid found in many dietary sources, including peanuts and chocolate. At least one report has shown that an oral nutritional supplement including arginine, omega fatty acids, and yeast RNA appeared to reduce postoperative inflammatory responses following cardiac surgery.

Glutamine is an alpha-amino acid that has been used as a nutritional supplement by bodybuilders for the relief of cramps and pain. Glutamine has also been shown to reduce postoperative healing time.

Aloe vera is an anti-inflammatory agent. Aloe vera is a succulent plant of the Lily family. The inner layer of the plant contains a clear gel. The gel is known to have an anti-inflammatory effect for the relief of pain.

Vitamin A and Vitamin D are both known to strengthen the immune system. Vitamin A has also been used in the treatment of cuts and scrapes, sprains and strains, and as an anti-inflammatory. Vitamin D is known to strengthen bones, particularly in osteoporosis.

Taurine (C₂H₇NO₃S) or 2-aminoethanesulfonic acid is a compound found in shellfish and organ meats. There is evidence that taurine is an inhibitory neurotransmitter in the central nervous system.

The composition is formulated for oral administration, such as a tablet, soft gel capsule, or the like, and may contain fillers, binders, and other excipients for the purpose. The above pain relief compositions have been found to be effective in alleviating pain for such conditions as a fractured vertebra, a ruptured disc, toothache, and arthritis, and is believed to be effective for the relief of pain resulting from virtually any disease or injury that produces pain.

It is important to note that in any of the formulations above iterated, the ingredient (MSM) may be eliminated for those allergic to sulfa, without reducing the efficacy of the composition(s). Similarly, fish oil and/or flax seed oil can be eliminated for those allergic to fish and nuts, respectively, again without compromising the efficacy of the formulation(s).

It is to be understood that the present invention is not limited to the embodiments described above, but encompasses any and all embodiments within the scope of the following claims.

I claim:

1. A pain relief composition, comprising:
at least one omega-3 fatty acid;
at least one antioxidant; and
at least one amino acid.

2. The pain relief composition according to claim 1, wherein said at least one omega-3 fatty acid comprises eicosapentaenoic acid (EPA).

3. The pain relief composition according to claim 1, wherein said at least one antioxidant comprises quercetin.

4. The pain relief composition according to claim 1, wherein said at least one antioxidant is selected from the group consisting of quercetin, rutin, and bioflavonoids other than quercetin and rutin.

5. The pain relief composition according to claim 1, wherein said at least one amino acid comprises DL-phenylalanine.

6. The pain relief composition according to claim 1, wherein said at least one amino acid comprises L-phenylalanine.

7. The pain relief composition according to claim 1, wherein said at least one amino acid is selected from the group consisting of L-glutamine, DL-phenylalanine, L-phenylalanine, histidine, valine, taurine, lysine, glycine, arginine, leucine and isoleucine.

8. The pain relief composition according to claim 1, wherein:
said at least one omega-3 fatty acid comprises about 10 mg to 6,000 mg of omega-3 fatty acids;
said at least one antioxidant comprises about 10 mg to 1,000 mg quercetin, about 10 mg to 1,000 mg rutin, and about 10 mg to 4,000 mg mixed bioflavonoids; and
said at least one amino acid comprises about 10 mg to 2,000 mg of at least one essential amino acid selected from the group consisting of L-glutamine, DL-phenylalanine, L-phenylalanine, histidine, valine, taurine, lysine, glycine, arginine, leucine, and isoleucine.

9. The pain relief composition according to claim 8, further comprising:
about 10 mg to 6,000 mg Vitamin C;
about 10 mg to 1,500 mg calcium;
about 10 mg to 1,000 mg magnesium;
about 5 mg to 50 mg zinc;
about 10 mg to 1,000 mg Vitamins B₁, B₂, B₆ and B₁₂;
about 50 mg to 3,000 mg of MSM; and
about 10 mg to 1,000 mg malic acid.

10. The pain relief composition according to claim 9, further comprising about 10 mg to 1,000 mg bromelain.

11. The pain relief composition according to claim 1, wherein:
said at least one omega-3 fatty acid comprises about 20 g to 6,000 mg of EPA/GLA;
said at least one antioxidant comprises about 10 mg to 1,000 mg quercetin, about 10 mg to 1,000 mg rutin, and about 10 mg to 4,000 mg mixed bioflavonoids; and
said at least one amino acid comprises about 10 mg to 2,000 mg of at least one essential amino acid selected from the group consisting of L-glutamine, DL-phenylalanine, histidine, valine, taurine, lysine, glycine, arginine, leucine, and isoleucine.

12. The pain relief composition according to claim 11, further comprising:
about 10 mg to 1,500 mg calcium;
about 10 mg to 1,000 mg magnesium;
about 5 mg to 50 mg zinc;
about 10 mg to 3,000 mg Vitamin C;
about 10 mg to 1,000 mg bromelain;
about 25 mg to 1,000 mg aloe vera gel;
about 5 IU to 5,000 IU Vitamin A;
about 5 IU to 500 IU Vitamin D; and
about 25 mg to 1,000 mg flax seed oil.

13. The pain relief composition according to claim 1, wherein:
said at least one omega-3 fatty acid comprises about 100 mg to 2,000 mg omega fatty acids selected from the group consisting of flaxseed oil and a combination of eicosapentaenoic acid (EPA) and gamma-linolenic acid (GLA);
said at least one antioxidant comprises about 500 mg quercetin, about 250 mg rutin, and about 1,000 mg of other mixed bioflavonoids;
said at least one amino acid comprises about 10 mg to 2,000 mg of DL-phenylalanine; the pain relief composition further comprising:
about 1,000 mg Vitamin C;
about 500 mg calcium;
about 250 mg magnesium;
about 15 mg zinc;
about 10 mg to 1,000 mg of Vitamins B₁, B₂, B₆ and B₁₂;
about 50 to 3,000 mg of (MSM); and
about 10 mg to 1,000 mg of malic acid.

14. The pain relief composition according to claim 1, wherein:
said at least one omega-3 fatty acid comprises about 1,000 mg to 2,000 mg omega fatty acids, such as flaxseed oil or a combination of eicosapentaenoic acid (EPA) and gamma-linolenic acid (GLA);
said at least one antioxidant comprises about 500 mg quercetin, about 250 mg rutin, and about 1,000 mg of other mixed bioflavonoids;
said at least one amino acid comprises about 10 mg to 2,000 mg of at least one essential amino acid selected from the group consisting of DL-phenylalanine and L-phenylalanine; the pain relief composition further comprising:
about 10 mg to 1,600 mg of bromelain;
about 500 mg to 1,000 mg Vitamin C;
about 500 mg calcium;
about 250 mg magnesium;
about 15 mg zinc;
about 10 mg to 1,000 mg of Vitamins B₁, B₂, B₆ and B₁₂;
about 50 mg to 3,000, mg of (MSM); and
about 10 mg to 1,000 mg of malic acid.

15. The pain relief composition according to claim 1, wherein:
said at least one omega-3 fatty acid comprises about 1,000 mg of a mixture of eicosapentaenoic acid (EPA) and gamma-linolenic acid (GLA);
said at least one antioxidant comprises about 500 mg quercetin, about 100 mg rutin, and about 1,000 mg of other mixed bioflavonoids;
said at least one amino acid comprises 500 mg DL-phenylalanine, about 500 mg arginine, about 250 mg glutamine, and about 100 mg taurine; the pain relief composition further comprising:
about 500 mg calcium;
about 250 mg magnesium;
about 10 mg zinc;
about 1,000 mg Vitamin C;
about 300 mg bromelain;
about 75 mg aloe vera gel;
about 200 IU Vitamin A;
about 50 IU Vitamin D; and
about 1,000 mg flax seed oil.

16. A pain relief composition, comprising:
about 10 mg to 6,000 mg of omega-3 fatty acids;
about 10 mg to 1,000 mg quercetin;
about 10 mg to 1,000 mg rutin;
about 10 mg to 4,000 mg mixed bioflavonoids;
about 10 mg to 6,000 mg Vitamin C;
about 10 mg to 1,500 mg calcium;
about 10 mg to 1,000 mg magnesium;
about 5 mg to 50 mg zinc;
about 10 mg to 1,000 mg Vitamins B₁, B₂, B₆ and B₁₂;
about 10 mg to 2,000 mg of at least one essential amino acid selected from the group consisting of L-glutamine, DL-phenylalanine, L-phenylalanine, histidine, valine, taurine, lysine, glycine, arginine, leucine, and isoleucine;
about 50 mg to 3,000 mg of MSM; and
about 10 mg to 1,000 mg malic acid.

17. The pain relief composition according to claim 16, further comprising about 10 mg to 1,000 mg bromelain.

18. A pain relief composition, comprising:
about 20 g to 6,000 mg of EPA/GLA or S-adenosylmethionine (SAM)/GLA;
about 10 mg to 1,500 mg calcium;
about 10 mg to 1,000 mg magnesium;
about 5 mg to 50 mg zinc;
about 10 mg to 3,000 mg Vitamin C;
about 10 mg to 1,000 mg quercetin;
about 10 mg to 1,000 mg rutin;
about 10 mg to 4,000 mg mixed bioflavonoids;
about 10 mg to 1,000 mg bromelain;
about 10 mg to 2,000 mg of at least one essential amino acid selected from the group consisting of L-glutamine, DL-phenylalanine, histidine, valine, taurine, lysine, glycine, arginine, leucine, and isoleucine;
about 25 mg to 1,000 mg aloe vera gel;
about 5 IU to 5,000 IU Vitamin A;
about 5 IU to 500 IU Vitamin D; and
about 25 mg to 1,000 mg flax seed oil.
